(19) United States
(12) Patent Application Publication
(10) Pub. No.: US 2012/0296011 A1
(43) Pub. Date: Nov. 22, 2012

(54) PHENOLIC COMPOUND, EPOXY RESIN, EPOXY RESIN COMPOSITION, PREPREG, AND CURED PRODUCT THEREOF

(75) Inventors: Kouichi Kawai, Kita-ku (JP); Katsuhiko Oshimi, Kita-ku (JP); Takao Sunaga, Kita-ku (JP); Kazuma Inoue, Kita-ku (JP)

(73) Assignee: NIPPONKAYAKU KABUSHIKIKAISHA, Chiyoda-ku, Tokyo (JP)

(21) Appl. No.: 13/575,661

(22) PCT Filed: Jan. 28, 2011
(86) PCT No.: PCT/JP2011/051832

§ 371 Date: Jul. 27, 2012

(30) Foreign Application Priority Data
Jan. 29, 2010 (JP) 2010-019269

(51) Int. Cl. C07C 45/74; C08L 63/00; C09D 163/00; C08G 65/40

(52) U.S. Cl. 523/400; 568/313; 528/212

(57) ABSTRACT

A phenolic compound which can be obtained by reacting the compound of the formula (1):

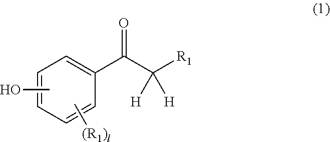

wherein R₁ groups are each independently present and represent a hydrogen atom etc. with the formula (6):

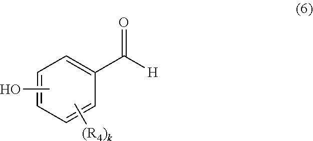

wherein R₄ groups are each independently present and represent a hydrogen atom etc.; and k represents the number of R₄ groups and is an integer of 0 to 4 and an epoxy resin which can be obtained by reacting the phenolic compound with an epihalohydrin are excellent in solvent solubility and also of which cured product has an excellent thermal conductance.

### TECHNICAL FIELD

The present invention relates to a novel phenolic compound, an epoxy resin, and an epoxy resin composition. Moreover, it relates to a cured product such as a prepreg formed of such an epoxy resin composition.

### BACKGROUND ART

Epoxy resin compositions are generally transformed into cured products which are excellent in mechanical properties, water resistance, chemical resistance, thermal resistance, electric properties, and the like and have been utilized in wide fields of adhesives, paints, laminated boards, forming materials, casting materials, and the like. In recent years, for the cured products of the epoxy resins to be used in these fields, high purity and also further improvement in various properties such as flame retardancy, thermal resistance, moisture resistance, toughness, low linear expansion coefficient, and low dielectric properties have been required.

Particularly, in electric and electronic industry fields that are representative uses of the epoxy resin compositions, high density mounting of semiconductors and high density wiring of printed wiring boards have been advanced for the purpose of multi-functionalization, improvement in performance, and compactification. However, with the high density mounting and high density wiring, heat generated from the inside of the semiconductor elements and printed wiring boards increases and may cause malfunction. Therefore, it becomes an important problem how the generated heat is efficiently released to the outside, from the viewpoint of energy efficiency and device design. As countermeasures against the heat, various contrivances such as use of metal core substrates, assembly of easily heat-releasable structures at the designing stage, and close packing of a high thermal conduction filler into a polymer material (epoxy resin) to be used have been performed. However, since the thermal conductivity of the polymer material as a binder which connects the high thermal conduction site is low, the thermal conduction speed of the polymer material determines the rate and thus it is a present situation that efficient heat release is not achieved.

As a method for realizing high thermal conduction of an epoxy resin, Patent Document 1 reports introduction of a mesogen group into the structure and the document describes an epoxy resin having a biphenyl skeleton as the epoxy resin having a mesogen group. Moreover, as an epoxy resin having a skeleton other than the biphenyl skeleton, a phenyl benzoate-type epoxy resin is described. However, it is necessary to produce the epoxy resin by an epoxidation reaction through oxidation, so that there are drawbacks in safety and cost and hence the method is not practical. As examples using epoxy resins having a biphenyl skeleton, Patent Documents 2 to 4 are mentioned and, of these, Patent Document 3 describes a method of using an inorganic filler having a high thermal conductivity in combination. However, the thermal conductance of the cured products obtained by the methods described in these documents is not a level which satisfies market demand. Therefore, there is desired an epoxy resin composition which uses an epoxy resin available at relatively low cost and affords a cured product having a higher thermal conductivity.

Moreover, many of highly thermal conductive epoxy resins having a mesogen group have a very high melting point; are difficult to take out in a resin form; and also are inferior in solvent solubility. Since such epoxy resins begin to cure before complete melting at curing, it is difficult to manufacture a homogeneous cured product and thus the resin is not preferable.

Furthermore, similarly to the epoxy resins, a curing agent to be contained in the epoxy resin composition is also an important element for realizing a high thermal conductance. Hitherto, as a curing agent contained in an epoxy resin composition whose cured product has a high thermal conductivity, there have been reported examples using an amine-based curing agent such as 4,4′-diaminodiphenylbenzoate or 4,4′-diaminodiphenylmethane in Patent Document 1 or 1,5-naphthalenediamine in Patent Documents 2 and 3. However, since these amine-based curing agents exhibit a curing acceleration action, it is difficult to assure a lifetime at preparing cured products and thus are not preferable. In Patent Document 4, catechol novolak is used as an example using a phenolic compound as the curing agent. However, the thermal conductivity of the cured product obtained by the method described in the document is also not a level which satisfies market demand and thus it is desired to develop an epoxy resin composition which affords a cured product having a higher thermal conductivity.

### BACKGROUND ART DOCUMENTS

### Patent Documents

Patent Document 1: JP-A-11-323162
Patent Document 2: JP-A-2004-2573
Patent Document 3: JP-A-2006-63315
Patent Document 4: JP-A-2003-137971

### SUMMARY OF THE INVENTION

### Problems that the Invention is to Solve

The present invention was accomplished as a result of investigation for solving such a problem and provides an epoxy resin which is excellent in solvent solubility; a cured product of which has a high thermal conductance; and a phenolic compound that is a precursor thereof.

### Means for Solving the Problems

As a result of extensive studies for solving the above problem, the inventors of the present invention have accomplished the present invention.

Namely, the present invention relates to:

(1) A phenolic compound obtained by reacting one or more compounds represented by the following formulae (1) to (5):

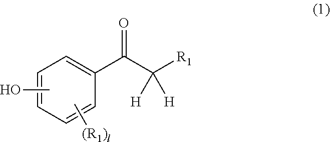

wherein R₁ groups are each independently present and represent any of a hydrogen atom, a substituted or unsubstituted alkyl group having 1 to 10 carbon atoms, a substituted or unsubstituted aryl group having 6 to 10 carbon atoms, a hydroxyl group, a nitro group, or a substituted or unsubstituted alkoxy group having 1 to 10 carbon atoms and 1 represents the number of R₁ groups and is an integer of 0 to 4;

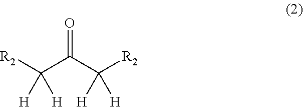

wherein R₂ groups are each independently present and represent any of a hydrogen atom, a substituted or unsubstituted alkyl group having 1 to 20 carbon atoms, a substituted or unsubstituted aryl group having 6 to 10 carbon atoms, a substituted or unsubstituted alkylcarbonyl group having 1 to 15 carbon atoms, a substituted or unsubstituted alkyl ester group having 2 to 10 carbon atoms, a substituted or unsubstituted alkoxy group having 1 to 10 carbon atoms, a morpholinylcarbonyl group, a phthalimido group, a piperonyl group, or a hydroxyl group;

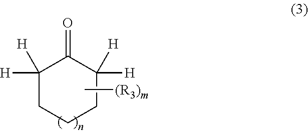

wherein R₃ groups are each independently present and represent any of a hydrogen atom, a substituted or unsubstituted alkylcarbonyl group having 0 to 10 carbon atoms, a substituted or unsubstituted alkyl group having 1 to 10 carbon atoms, a substituted or unsubstituted aryl group having 6 to 10 carbon atoms, a substituted or unsubstituted alkyl ester group having 2 to 10 carbon atoms, a substituted or unsubstituted alkoxy group having 1 to 10 carbon atoms, or a hydroxyl group; n represents the number of carbon atoms and is any integer of 0, 1 and 2; and m represents the number of R₃ groups and satisfies the relation: 0≦m≦n+2;

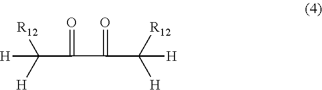

wherein R₁₂ groups are each independently present and represent any of a hydrogen atom, a substituted or unsubstituted alkyl group having 1 to 20 carbon atoms, a substituted or unsubstituted aryl group having 6 to 10 carbon atoms, a substituted or unsubstituted alkoxy group having 1 to 10 carbon atoms, or a hydroxyl group;

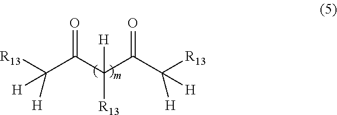

wherein R₁₃ groups are each independently present and represent any of a hydrogen atom, a substituted or unsubstituted alkyl group having 1 to 20 carbon atoms, a substituted or unsubstituted aryl group having 6 to 10 carbon atoms, a substituted or unsubstituted alkoxy group having 1 to 10 carbon atoms, a substituted or unsubstituted alkyl ester group having 1 to 10 carbon atoms, or a hydroxyl group; and m is an integer of 1 to 10; with a compound of the following formula (6):

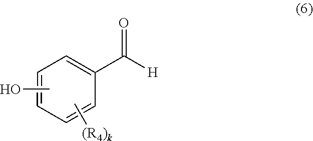

wherein R₄ groups are each independently present and represent any of a hydrogen atom, a substituted or unsubstituted alkyl group having 1 to 10 carbon atoms, a substituted or unsubstituted aryl group having 6 to 10 carbon atoms, a hydroxyl group, a nitro group, a formyl group, an allyl group, or a substituted or unsubstituted alkoxy group having 1 to 10 carbon atoms; and k represents the number of R₄ groups and is an integer of 0 to 4;

(2) An epoxy resin obtained by reacting the phenolic compound described in the above (1) with an epihalohydrin;

(3) The epoxy resin according to the above (2), wherein total halogen content is 1800 ppm or less;

(4) An epoxy resin composition comprising at least one of the epoxy resin described in the above (2) or (3) and the phenolic compound described in the above (1);

(5) The epoxy resin composition according to the above (4), which contains an inorganic filler having a thermal conductivity of 20 W/m·K or more;

(6) The epoxy resin composition according to the above (4) or (5), which is used in semiconductor encapsulation uses;

(7) A prepreg comprising the epoxy resin composition described in the above (4) or (5) and a sheet-shaped fiber substrate;

(8) A cured product obtained by curing the epoxy resin composition described in any one of the above (4) to (6) or the prepreg according to (7);

(9) A process for producing the epoxy resin composition according to the above (3), wherein flaky sodium hydroxide is added into a reaction system at the reaction of the phenolic compound with the epihalohydrin;

(10) The process according to the above (9), wherein the flaky sodium hydroxide is added into the reaction system for multiple times;

(11) The process according to the above (9) or (10), wherein the epihalohydrin is used in an amount of 2 to 15 mol based on 1 mol of the hydroxyl group of the phenolic compound; and

(12) The process according to the above (9) or (10), wherein the epihalohydrin is used in an amount of 2 to 4.5 mol based on 1 mol of the hydroxyl group of the phenolic compound.

### Effect of the Invention

The phenolic compound and epoxy resin of the present invention are useful in the case where they are used in semiconductor-encapsulating materials, various composite materials including prepregs, adhesives, paints, and the like since cured products thereof are excellent in thermal conductance. Moreover, since the epoxy resin of the present invention has a low melting point as compared with an epoxy resin having a mesogen group and further is also excellent in solvent solubility, a homogeneous cured product can be obtained.

### MODE FOR CARRYING OUT THE INVENTION

First, the phenolic compound of the present invention will be explained. The phenolic compound of the present invention is obtained by reacting one or more compound selected from the compounds represented by the following formulae (1) to (5) with the compound represented by the following formula (6):

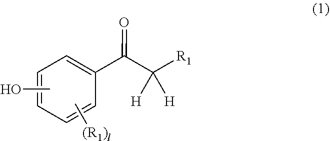

wherein R₁ groups are each independently present and represent any of a hydrogen atom, a substituted or unsubstituted alkyl group having 1 to 10 carbon atoms, a substituted or unsubstituted aryl group having 6 to 10 carbon atoms, a hydroxyl group, a nitro group, or a substituted or unsubstituted alkoxy group having 1 to 10 carbon atoms and 1 represents the number of R₁ groups and is an integer of 0 to 4.

In the formula (1), R₁ groups are each independently present and are preferably a hydrogen atom, an unsubstituted alkyl group having 1 to 10 carbon atoms, an unsubstituted aryl group having 6 to 10 carbon atoms, a hydroxyl group, a nitro group, or an unsubstituted alkoxy group having 1 to 10 carbon atoms.

Specific examples of the compound represented by the formula (1) to be used in the reaction with the compound represented by the formula (6) for obtaining the phenolic compound of the present invention include 2-hydroxyacetophenone, 3-hydroxyacetophenone, 4-hydroxyacetophenone, 2′,4′-dihydroxyacetophenone, 2′,5′-dihydroxyacetophenone, 3′,4′-dihydroxyacetophenone, 3′,5′-dihydroxyacetophenone, 2′,3′,4′-trihydroxyacetophenone, 2′,4′,6′-trihydroxyacetophenone monohydrate, 4′-hydroxy-3′-methylacetophenone, 4′-hydroxy-2′-methylacetophenone, 2′-hydroxy-5′-methylacetophenone, 4′-hydroxy-3′-methoxyacetophenone, 2′-hydroxy-4′-methoxyacetophenone, 4′-hydroxy-3′-nitroacetophenone, 4′-hydroxy-3′,5′-dimethoxyacetophenone, 4′,6′-dimethoxy-2′-hydroxyacetophenone, 2′-hydroxy-3′,4′-dimethoxyacetophenone, 2′-hydroxy-4′,5′-dimethoxyacetophenone, methyl 5-acetylsalicylate, 2′,3′-dihydroxy-4′-methoxyacetophenone hydrate. Of these, 4′-hydroxy-3′-methoxyacetophenone and 4′-hydroxyacetophenone are preferred since they have a high solvent solubility at the time when the resulting phenolic compound is epoxidized and a cured product of the epoxy resin composition exhibits a high thermal conductance.

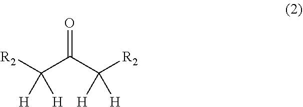

wherein R₂ groups are each independently present and represent any of a hydrogen atom, a substituted or unsubstituted alkyl group having 1 to 20 carbon atoms, a substituted or unsubstituted aryl group having 6 to 10 carbon atoms, a substituted or unsubstituted alkylcarbonyl group having 1 to 15 carbon atoms, a substituted or unsubstituted alkyl ester group having 2 to 10 carbon atoms, a substituted or unsubstituted alkoxy group having 1 to 10 carbon atoms, a morpholinylcarbonyl group, a phthalimido group, a piperonyl group, or a hydroxyl group.

In the formula (2), the above substituent is preferably at least one group selected from the group consisting of a carbonyl group, an ester group, an alkenyl group, a phenyl group, an ether group, a phthalimido group, and a piperonyl group.

Specific examples of the compound represented by the formula (2) to be used in the reaction with the compound represented by the formula (6) for obtaining the phenolic compound of the present invention include acetone, 1,3-diphenyl-2-ppropanone, 2-butanone, 1-phenyl-1,3-butanedione, 2-pentanone, 3-pentanone, 4-methyl-2-pentanone, acetylacetaone, 2-hexanone, 3-hexanone, isoamyl methyl ketone, ethyl isobutyl ketone, 4-methyl-2-hexanone, 2,5-hexanedione, 1,6-diphenyl-1,6-hexanedione, 2-heptanone, 3-heptanone, 4-heptanone, 2-methyl-4-heptanone, 5-methyl-3-heptanone, 6-methyl-2-heptanone, 2,6-dimethyl-4-heptanone, 2-octanone, 3-octanone, 4-octanone, 5-methyl-2-octanone, 2-nonanone, 3-nonanone, 4-nonanone, 5-nonanone, 2-decanone, 3-decanone, 4-decanone, 5-decanone, 2-undecanone, 3-undecanone, 4-undecanone, 5-undecanone, 6-undecanone, 2-methyl-4-undecanone, 2-dodecanone, 3-dodecanone, 4-dodecanone, 5-dodecanone, 6-dodecanone, 2-tetradecanone, 3-tetradecanone, 8-pentadecanone, 10-nonadecanone, 7-tridecanone, 2-pentadecanone, 3-hexadecanone, 9-heptadecanone, 11-heneicosanone, 12-tricosanone, 14-heptacosanone, 16-hentriacontanone, 18-pentatriacontanone, 4-ethoxy-2-butanone, 4-(4-methoxyphenyl)-2-butanone, 4-methoxy-4-methyl-2-pentanone, 4-methoxyphenylacetone, methoxyacetone, phenoxyacetone, methyl acetoacetate, ethyl acetoacetate, propyl acetoacetate, butyl acetoacetate, isobutyl acetoacetate, sec-butyl acetoacetate, tert-butyl acetoacetate, 3-pentyl acetoacetate, amyl acetoacetate, isoamyl acetoacetate, hexyl acetoacetate, heptyl acetoacetate, n-octyl acetoacetate, benzyl acetoacetate, dimethyl acetylsuccinate, dimethyl acetonylmalonate, diethyl acetonylmalonate, 2-methoxyethyl acetoacetate, allyl acetoacetate, 4-sec-butoxy-2-butanone, benzyl butyl ketone, bisdemethoxycurcumin, 1,1-dimethoxy-3-butanone, 1,3-diacetoxyacetone, 4-hydroxyphenylacetone, 4-(4-hydroxyphenyl)-2-butanone, isoamyl methyl ketone, 4-hydroxy-2-butanone, 5-hexen-2-one, acetonylacetone, 3,4-dimethoxyphenylacetone, piperonyl methyl ketone, piperonylacetone, phthalimidoacetone, 4-isopropoxy-2-butanone, 4-isobutoxy-2-butanone, acetoxy-2-propanone, N-acetoacetylmorpholine, 1-acetyl-4-piperidone, and the like. Of these, acetone is preferable since it has a high solvent solubility at the time when the resulting phenolic compound is epoxidized and a cured product of the epoxy resin composition exhibits a high thermal conductance.

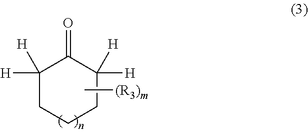

wherein R₃ groups are each independently present and represent any of a hydrogen atom, a substituted or unsubstituted alkylcarbonyl group having 0 to 10 carbon atoms, a substituted or unsubstituted alkyl group having 1 to 10 carbon atoms, a substituted or unsubstituted aryl group having 6 to 10 carbon atoms, a substituted or unsubstituted alkyl ester group having 2 to 10 carbon atoms, a substituted or unsubstituted alkoxy group having 1 to 10 carbon atoms, or a hydroxyl group; n represents the number of carbon atoms and is any integer of 0, 1 and 2; and m represents the number of R₃ groups and satisfies the relation: 0≦m≦n+2.

In this regard, in the formula (3), the case where R₃ is a substituted or unsubstituted alkylcarbonyl group having 0 carbon atom represents a carbonyl structure including a carbon atom constituting the cycloalkane which is a main skeleton of the formula (3).

In the formula (3), it is preferable that the above substituent is an ether group or a carbonyl group.

Specific examples of the compound represented by the formula (3) to be used in the reaction with the compound represented by the formula (6) for obtaining the phenolic compound of the present invention include cyclopentanone, 3-phenylcyclopentanone, 2-acetylcyclopentanone, 1,3-cyclopentanedione, 2-methyl-1,3-cyclopentanedione, 2-ethyl-1,3-cyclopentanedione, cyclohexanone, 3-methylcyclohexanone, 4-methylcyclohexanone, 4-ethylcyclohexanone, 4-tert-butylcyclohexanone, 4-pentylcyclohexanone, 3-phenylcyclohexanone, 4-phenylcyclohexanone, 3,3-dimethylcyclohexanone, 3,4-dimethylcyclohexanone, 3,5-dimethylcyclohexanone, 4,4-dimethylcyclohexanone, 3,3,5-trimethylcyclohexanone, 2-acetylcyclohexanone, ethyl 4-cyclohexanonecarboxylate, 1,4-cyclohexanedione monoethylene ketal, bicyclohexane-4,4′-dione monoethylene ketal, 1,4-cyclohexanedione mono-2,2-dimethyltrimethylene ketal, 2-acetyl-5,5-dimethyl-1,3-cyclohexanedione, 1,2-cyclohexanedione, 1,3-cyclohexanedione, 1,4-cyclohexanedione, 2-methyl-1,3-cyclohexanedione, 5-methyl-1,3-cyclohexanedione, dimedone, dimethyl 1,4-cyclohexanedione-2,5-dicarboxylate, 4,4′-bicyclohexanone, 2,2-bis(4-oxocyclohexyl)propane, cycloheptanone, and the like. Of these, cyclopentanone, cyclohexanone, cycloheptanone, and 4-methylcyclohexanone are preferable since they have a high solvent solubility at the time when the resulting phenolic compound is epoxidized and a cured product of the epoxy resin composition exhibits a high thermal conductance.

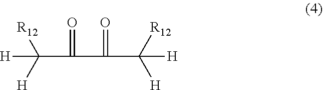

wherein R₁₂ groups are each independently present and represent any of a hydrogen atom, a substituted or unsubstituted alkyl group having 1 to 20 carbon atoms, a substituted or unsubstituted aryl group having 6 to 10 carbon atoms, a substituted or unsubstituted alkoxy group having 1 to 10 carbon atoms, or a hydroxyl group.

In the formula (4), R₁₂ groups are each independently present and preferably a hydrogen atom, a substituted or unsubstituted alkyl group having 1 to 20 carbon atoms, a substituted or unsubstituted aryl group having 6 to 10 carbon atoms, a substituted or unsubstituted alkoxy group having 1 to 10 carbon atoms, or a hydroxyl group.

Specific examples of the compound represented by the formula (4) to be used in the reaction with the compound represented by the formula (6) for obtaining the phenolic compound of the present invention include diacetyl, 2,3-pentanedione, 3,4-hexanedione, 5-methyl-2,3-hexanedione, 2,3-heptanedione, and the like. Of these, diacetyl is preferable since it has a high solvent solubility at the time when the resulting phenolic compound is epoxidized and a cured product of the epoxy resin composition exhibits a high thermal conductance.

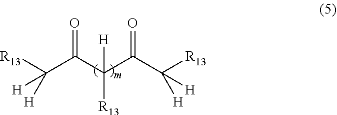

wherein R₁₃ groups are each independently present and represent any of a hydrogen atom, a substituted or unsubstituted alkyl group having 1 to 20 carbon atoms, a substituted or unsubstituted aryl group having 6 to 10 carbon atoms, a substituted or unsubstituted alkoxy group having 1 to 10 carbon atoms, a substituted or unsubstituted alkyl ester group having 1 to 10 carbon atoms, or a hydroxyl group; and m is an integer of 1 to 10.

In the formula (5), R₁₃ groups are each independently present and preferably a hydrogen atom, a substituted or unsubstituted alkyl group having 1 to 20 carbon atoms, a substituted or unsubstituted aryl group having 6 to 10 carbon atoms, a substituted or unsubstituted alkoxy group having 1 to 10 carbon atoms, a substituted or unsubstituted alkyl ester group having 1 to 10 carbon atoms, or a hydroxyl group.

Specific examples of the compound represented by the formula (5) to be used in the reaction with the compound represented by the formula (6) for obtaining the phenolic compound of the present invention include ethyl diacetoacetate, 2,5-hexanedione, 3-methyl-2,4-pentanedione, 3-ethyl-2,4-pentanedione, 3-butyl-2,4-pentanedione, 3-phenyl-2,4-pentanedione, ethyl 4-acetyl-5-oxohexanoate, and the like. Of these, 3-methyl-2,4-pentanedione and 3-ethyl-2,4-pentanedione are preferable since they have a high solvent solubility at the time when the resulting phenolic compound is epoxidized and a cured product of the epoxy resin composition exhibits a high thermal conductance.

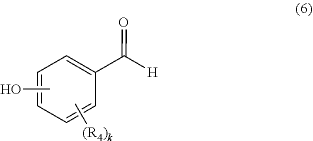

wherein R₄ groups are each independently present and represent any of a hydrogen atom, a substituted or unsubstituted alkyl group having 1 to 10 carbon atoms, a substituted or unsubstituted aryl group having 6 to 10 carbon atoms, a hydroxyl group, a nitro group, a formyl group, an allyl group, or a substituted or unsubstituted alkoxy group having 1 to 10 carbon atoms and k represents the number of R₄ groups and is an integer of 0 to 4.

Specific examples of the compound represented by the formula (6) to be used in the reaction with one or more compounds selected from the compounds represented by the formulae (1) to (5) for obtaining the phenolic compound of the present invention include 2-hydroxybenzaldehyde, 3-hydroxybenzaldehyde, 4-hydroxybenzaldehyde, 2,3-dihydroxybenzaldehyde, 2,4-dihydroxybenzaldehyde, 2,5-dihydroxybenzaldehyde, 3,4-dihydroxybenzaldehyde, syringaaldehyde, 3,5-di-tert-butyl-4-hydroxybenzaldehyde, isovanilline, 4-hydroxy-3-nitrobenzaldehyde, 5-hydroxy-2-nitrobenzaldehyde, 3,4-dihydroxy-5-nitrobenzaldehyde, vanillin, o-vanillin, 2-hydroxy-1-naphthoaldehyde, 2-hydroxy-5-nitro-m-anisaldehyde, 2-hydroxy-5-methylisophthalaldehyde, 2-hydroxy-4-methoxybenzaldehyde, 1-hydroxy-2-naphthoaldehyde, 2-hydroxy-5-methoxybenzaldehyde, 5-nitrovanillin, 5-allyl-3-methoxysalicylaldehyde, 3,5-di-tert-butylsalicylaldehyde, 3-ethoxysalicylaldehyde, 4-hydroxyisophthalaldehyde, 4-hydroxy-3,5-dimethylbenzaldehyde, 2,4,6-trihydroxybenzaldehyde, 2,4,5-trihydroxybenzaldehyde, 2,3,4-trihydroxybenzaldehyde, 3,4,5-trihydroxybenzaldehyde, 3-ethoxy-4-hydroxybenzaldehyde, and the like. They may be used singly or two or more thereof may be used in combination. Of these, vanillin is preferably used singly since it has a high solvent solubility at the time when the resulting phenolic compound is epoxidized and a cured product of the epoxy resin composition exhibits a high thermal conductance.

The phenolic compound of the present invention is obtained by aldol condensation reaction of one or more of the compounds represented by the formulae (1) to (5) with the compound represented by the formula (6) under an acidic condition or a basic condition.

The compound represented by the formula (6) is used in an amount of 1.0 to 1.05 mol based on 1 mol of the compound represented by the formula (1) and in an amount of 2.0 to 3.15 mol based on 1 mol of the compounds represented by the formulae (2), (3), (4), and (5).

In the case where the aldol condensation reaction is carried out under an acidic condition, usable acidic catalyst includes inorganic acids such as hydrochloric acid, sulfuric acid, and nitric acid and organic acids such as toluenesulfonic acid, xylenesulfonic acid, and oxalic acid. They may be used singly or plural kinds thereof may be used in combination. The amount of the acidic catalyst to be used is from 0.01 to 1.0 mol, preferably from 0.2 to 0.5 mol based on 1 mol of the compound represented by the formula (6).

On the other hand, in the case where the aldol condensation reaction is carried out under a basic condition, usable basic catalyst includes metal hydroxides such as sodium hydroxide and potassium hydroxide, alkali metal carbonate salts such as potassium carbonate and sodium carbonate, amine derivatives such as diethylamine, triethylamine, tributylamine, diisobutylamine, pyridine, and piperidine, and amino alcohol derivatives such as dimethylaminoethyl alcohol and diethylaminoethyl alcohol. Also, in the case of the basic condition, the basic catalysts mentioned above may be used singly or plural kinds thereof may be used in combination. The amount of the basic catalyst to be used is from 0.1 to 2.5 mol, preferably from 0.2 to 2.0 mol based on 1 mol of the compound represented by the formula (6).

In the reaction for obtaining the phenolic compound of the present invention, a solvent may be used according to need. Although usable solvent is not particularly limited unless it has reactivity with the compound represented by the formula (6), for example, such as ketones, it is preferred to use an alcohol as a solvent in view of easily dissolving the compound represented by the formula (6) that is a raw material.

The reaction temperature is usually from 10 to 90° C., preferably from 35 to 70° C. The reaction time is usually from 0.5 to 10 hours. However, since reactivity is different depending on the kind of the starting compound, it is not limited thereto. After completion of the reaction, in the case where the product is taken out as a resin, after washing the reaction product with water or without washing with water, unreacted matter, the solvent, and the like are removed from the reaction solution under heating and reduced pressure. In the case where the product is taken out as crystals, the crystals are precipitated by adding the reaction solution dropwise into a large amount of water. Since the formed phenolic compound of the present invention may be dissolved into water in the case where the reaction is carried out under a basic condition, the product is precipitated as crystals with making the condition neutral to acidic by adding hydrochloric acid or the like.

The following will explain the epoxy resin of the present invention.

The epoxy resin of the present invention is obtained by reacting the phenolic compound of the present invention obtained by the above method with an epihalohydrin to form an epoxide. At the epoxidation, only one kind of the phenolic compound of the present invention may be used or two or more kinds thereof may be used in combination. Moreover, the phenolic compound of the present invention may be used in combination with a phenolic compound other than the phenolic compound of the present invention.

As the phenolic compound which can be used in combination other than the phenolic compound of the present invention, any one can be used without particular limitation as long as it is a phenolic compound usually used as a raw material of an epoxy resin. However, since there is a concern of impairing the effect of the present invention that a cured product has a high thermal conductivity, the amount of the phenolic compound usable in combination is preferably as small as possible and it is particularly preferred to use only the phenolic compound of the present invention.

As the epoxy resin of the present invention, since a cured product exhibiting a particularly excellent solvent solubility and also having a high thermal conductivity is obtained, an epoxidized compound of the phenolic compound of the present invention obtained by reacting the compound represented by the formula (6) with the compound represented by the formula (3) is preferable.

In the reaction of obtaining the epoxy resin of the present invention, as the epihalohydrin, epichlorohydrin, α-methylepichlorohydrin, β-methylepichlorohydrin, epibromohydrin, and the like can be used and epichlorohydrin, which is industrially easily available, is preferable. The amount of the epihalohydrin to be used is usually from 2 to 20 mol, preferably from 2 to 15 mol and particularly preferably from 2 to 4.5 mol based on 1 mol of the hydroxyl group of the phenolic compound of the present invention. The epoxy resin is obtained by adding the phenolic compound to the epihalohydrin in the presence of an alkali metal oxide and then ring-opening the formed 1,2-halohydrin ether group to achieve epoxidation. On this occasion, by using the epihalohydrin in a remarkably smaller amount than the usual amount as above, the molecular weight of the epoxy resin can be enlarged and also the molecular weight distribution can be broadened. As a result, the resulting epoxy resin can be taken out as a resinous product having a relatively low softening point from the system and exhibits an excellent solvent solubility.

The alkali metal hydroxide which can be used in the epoxidation reaction includes sodium hydroxide, potassium hydroxide, and the like and they may be used as they are or may be used as an aqueous solution thereof. In the case of using the aqueous solution, a method of continuously adding the aqueous solution of the alkali metal hydroxide into the reaction system, also continuously removing water by liquid separation from a mixed solution of water and the epihalohydrin continuously distilled under reduced pressure or under ordinary pressure, and continuously returning the epohalohydrin alone into the reaction system may be used. The amount of the alkali metal hydroxide to be used is usually from 0.9 to 3.0 mol; preferably from 1.0 to 2.5 mol; more preferably from 1.0 to 2.0 mol; and particularly preferably from 1.0 to 1.3 mol based on 1 mol of the hydroxyl group of the phenolic compound of the present invention.

Moreover, the inventors of the present invention have found that it becomes possible to remarkably reduce the amount of halogen contained in the resulting epoxy resin by the use of particularly flaky sodium hydroxide in the epoxidation reaction in comparison with the use of sodium hydroxide as an aqueous solution. The halogen is derived from the epihalohydrin and the higher amount thereof mixed into the epoxy resin causes more decrease in thermal conductance of the cured product. Furthermore, the flaky sodium hydroxide is preferably added into the reaction system by portions. By the addition thereof by portions, the reaction temperature can be prevented from rapidly decreasing. Thereby, the formation of a 1,3-halohydrin compound and a halomethylene compound as impurities can be prevented and thus it becomes possible to form a cured product having a higher thermal conductivity.

In order to accelerate the epoxidation reaction, it is preferable to add a quaternary ammonium salt such as tetramethylammonium chloride, tetramethylammonium bromide, or trimethylbenzylammonium chloride as a catalyst. The amount of the quaternary ammonium salt to be used is usually from 0.1 to 15 g; and preferably from 0.2 to 10 g based on 1 mol of the hydroxyl group of the phenolic compound of the present invention.

Moreover, at the epoxidation, it is preferable to carry out the reaction after adding an alcohol such as methanol, ethanol, or isopropyl alcohol or an aprotic polar solvent such as dimethyl sulfone, dimethyl sulfoxide, tetrahydrofuran, or dioxane, in view of proceeding of the reaction. Of these, an alcohol or dimethyl sulfoxide is preferable. In the case of using an alcohol, the epoxy resin can be obtained in high yields. On the other hand, in the case of using dimethyl sulfoxide, the amount of the halogen in the epoxy resin can be further reduced.

In the case of using the alcohol, the amount thereof is usually from 2 to 50% by mass, preferably from 4 to 35% by mass based on the amount of the epihalohydrin used. Moreover, in the case of using the aprotic polar solvent, the amount thereof is usually from 5 to 100% by mass and preferably from 10 to 80% by mass based on the amount of the epihalohydrin used.

The reaction temperature is usually from 30 to 90° C., preferably from 35 to 80° C. The reaction time is usually from 0.5 to 10 hours, preferably from 1 to 8 hours.

After completion of the reaction, the epihalohydrin, the solvent, and the like are removed from the reaction solution under heating and reduced pressure after washing the reaction mixture with water or without washing with water. Moreover, in order to further reduce the amount of the halogen contained in the epoxy resin, it is also possible to assure the ring-closure by dissolving the recovered epoxy resin of the present invention into a solvent such as toluene or methyl isobutyl ketone and carrying out the reaction with adding an aqueous solution of an alkali metal hydroxide such as sodium hydroxide or potassium hydroxide. In this case, the amount of the alkali metal hydroxide is usually from 0.1 to 0.3 mol, preferably from 0.05 to 2 mol based on 1 mol of the hydroxyl group of the phenolic compound of the present invention. The reaction temperature is usually from 50 to 120° C. and the reaction time is usually from 0.5 to 2 hours.

After the completion of the reaction, the epoxy resin of the present invention is obtained by removing formed salt by filtration, washing with water, or the like and further removing the solvent by distillation under heating and reduced pressure. Moreover, in the case where the epoxy resin of the present invention precipitates as crystals, the crystals of the epoxy resin of the present invention can be collected by filtration after dissolving the formed salt in a large amount of water.

The total halogen content in the epoxy resin of the present invention which is obtained using flaky sodium hydroxide as mentioned above is usually 1800 ppm or less; preferably 1600 ppm or less; and further preferably 700 ppm or less. When the total halogen content is too large, the halogen remains as an uncrosslinked terminal in addition to the fact that an adverse influence is exerted on electric reliability of the cured product, so that orientation of the molecules one another in the melted state at curing does not proceed and the situation leads to decrease in thermal conductance.

The epoxy resin composition of the present invention is described below. The epoxy resin composition of the present invention contains at least one of the epoxy resin of the present invention and the phenolic compound of the present invention as an essential component.

In the epoxy resin composition of the present invention, the epoxy resin of the present invention can be used singly or may be used in combination with the other epoxy resin.

Specific examples of the other epoxy resin include polycondensates of bisphenols (bisphenol A, bisphenol F, bisphenol S, bisphenol, bisphenol AD, bisphenol I, etc.) or phenols (phenol, an alkyl-substituted phenol, an aromatic group-substituted phenol, naphthol, an alkyl-substituted naphthol, dihydroxybenzene, an alkyl-substituted dihydroxybenzene, dihydroxynaphthalene, etc.) with various aldehydes (formaldehyde, acetaldehyde, an alkylaldehyde, benzaldehyde, an alkyl-substituted benzaldehyde, hydroxybenzaldehyde, naphthaldehyde, glutaraldehyde, phthalaldehyde, crotonaldehyde, cinnamaldehyde, etc.); polycondensates of a polycondensate of an aromatic compound such as xylene and formaldehyde with phenols; polymerized products of phenols with various diene compounds (dicyclopentadiene, a terpene, vinylcyclohexene, norbornadiene, vinylnorbornene, tetrahydroindene, divinylbenzene, divinylbiphenyl, diisopropenylbiphenyl, butadiene, isoprene, etc.); polycondensates of phenols with ketones (acetone, methyl ethyl ketone, methyl isobutyl ketone, acetophenone, benzophenone, etc.); polycondensates of phenols with aromatic dimethanols (benzenedimethanol, biphenyldimethanol, etc.); polycondensates of phenols with aromatic dichloromethyls (α,α′-dichloroxylene, bischloromethylbiphenyl, etc.); polycondensates of phenols with aromatic bisalkoxymethyls (bismethoxymethylbenzene, bismethoxymethylbiphenyl, bisphenoxymethylbiphenyl, etc.); polycondensates of bisphenols with various aldehydes; and glycidyl ether-based epoxy resins in which alcohols are glycidylated, alicyclic epoxy resins, glycidylamine-based epoxy resins, glycidyl ester-based epoxy resins, and the like. However, examples are not limited thereto as long as they are usually used epoxy resins. They may be used singly or two or more kinds thereof may be used in combination.

In the case of using the other epoxy resin in combination, with regard to the ratio of the epoxy resin of the present invention in the total epoxy resin components in the epoxy resin composition of the present invention, 30% by mass or more is preferable; 40% by mass or more is more preferable; 70% by mass or more is further more preferable; 100% by mass (the case where the other epoxy resin is not used in combination) is particularly preferable. However, in the case where the epoxy resin of the present invention is used as a modifying agent of the epoxy resin composition, it is added in a ratio of 1 to 30% by mass in the total epoxy resin.

The phenolic compound of the present invention is contained in the epoxy resin composition of the present invention as a curing agent. The curable resin in this case may be the epoxy resin of the present invention mentioned above or may be another epoxy resin other than it.

In the epoxy resin composition of the present invention, the phenolic compound of the present invention can be used singly or in combination with the other curing agent.

Examples of the other curing agent contained in the epoxy resin composition of the present invention include amine-based compounds, acid anhydride-based compounds, amide-based compounds, phenol-based compounds, and the like. Specific examples of these other curing agents are shown in the following (a) to (e).

(a) Amine-based compounds: diaminodiphenylmethane, diethylenetriamine, triethylenetetramine, diaminodiphenyl sulfone, isophoronediamine, naphthalenediamine, and the like;

(b) Acid anhydride-based compounds: phthalic anhydride, trimellitic anhydride, pyromellitic anhydride, maleic anhydride, tetrahydrophthalic anhydride, methyltetrahydrophthalic anhydride, methylnadic anhydride, hexahydrophthalic anhydride, methylhexahydrophthalic anhydride, and the like;

(c) Amide-based compounds: dicyandiamide, a polyamide resin synthesized from a dimer of linolenic acid and ethylenediamine, or the like;

(d) Phenol-based compounds: polyhydric phenols (bisphenol A, bisphenol F, bisphenol S, fluorene bisphenol, terpene diphenol, 4,4′-dihydroxybiphenyl, 2,2′-dihydroxybiphenyl, 3,3′,5,5′-tetramethyl-(1,1′-biphenyl)-4,4′-diol, hydroquinone, resorcin, naphthalenediol, tris-(4-hydroxyphenyl)methane, and 1,1,2,2-tetrakis(4-hydroxyphenyl)ethane, etc.); phenolic resins obtained by condensation of phenols (e.g., phenol, an alkyl-substituted phenol, naphthol, an alkyl-substituted naphthol, dihydroxybenzene, dihydroxynaphthalene, etc.) with aldehydes (formaldehyde, acetaldehyde, benzaldehyde, p-hydroxybenzaldehyde, o-hydroxybenzaldehyde, furfural, etc.), ketones (p-hydroxyacetophenone, o-hydroxyacetophenone, etc.), or dienes (dicyclopentadiene, tricyclopentadiene, etc.); phenolic resins obtained by polycondensation of the above phenols with substituted biphenyls (4,4′-bis(chloromethyl)-1,1′-biphenyl, 4,4′-bis(methoxymethyl)-1,1′-biphenyl, etc.), substituted phenyls (1,4-bis(chloromethyl)benzene, 1,4-bis(methoxymethyl)benzene, 1,4-bis(hydroxymethyl)benzene, etc.), or the like; modified products of the above phenols and/or the above phenolic resins; halogenated phenols such as tetrabromobisphenol A and brominated phenolic resins;

(e) Others: imidazoles, BF₃-amine complexes, guanidine derivatives.

Among these other curing agents, curing agents having a structure in which an active hydrogen is adjacent, for example, amine-based compounds such as diaminodiphenylmethane, diaminodiphenyl sulfone, and naphthalenediamine, condensates of catechol with aldehydes, ketones, dienes, substituted biphenyls, or substituted phenyls, and the like, are preferable since they contribute arrangement of the epoxy resin.

The other curing agents may be used singly or a plurality thereof may be used in combination. In the case of using the other curing agent in combination, with regard to the ratio of the phenolic compound of the present invention in the total curing agent components in the epoxy resin composition of the present invention, 20% by mass or more is preferable; 30% by mass or more is more preferable; 70% by mass or more is further more preferable; and 100% by mass is particularly preferable (the case where the other curing agent is not used in combination).

In the epoxy resin composition of the present invention, the amount of the total curing agents including the phenolic compound of the present invention to be used is preferably from 0.5 to 2.0 equivalents; and particularly preferably from 0.6 to 1.5 equivalents to 1 equivalent of the epoxy group of the whole epoxy resin.

As the epoxy resin composition of the present invention, the case where the epoxy resin of the present invention is used in a ratio of 100% by mass as the epoxy resin and the phenolic compound of the present invention is used in a ratio of 100% as the curing agent is most preferable.

By including an inorganic filler which is excellent in thermal conductance to the epoxy resin composition of the present invention according to need, a further excellent high thermal conductance can be imparted to a cured product thereof.

The inorganic filler to be contained in the epoxy resin composition of the present invention is added for the purpose of imparting a higher thermal conductivity to the cured product of the epoxy resin composition. In the case where the thermal conductivity of the inorganic filler itself is exceedingly low, there is a concern that the high thermal conductivity obtained by the combination of the epoxy resin and the curing agent is impaired. Therefore, as the inorganic filler to be contained in the epoxy resin composition of the present invention, one having a higher thermal conductivity is more preferable and there is no limitation as long as it has a thermal conductivity of usually 20 W/m·K or more; preferably 30 W/m·K or more; and more preferably 50 W/m·K or more. In this regard, the thermal conductivity herein is a value measured by the method in accordance with ASTM E1530. Examples of the inorganic filler having such a property include inorganic powder fillers such as boron nitride, aluminum nitride, silicon nitride, silicon carbide, titanium nitride, zinc oxide, tungsten carbide, alumina, and magnesium oxide; fibrous fillers such as synthetic fibers and ceramic fibers; colorants; and the like. The shape of these inorganic fillers may be any of powders (block, sphere), single fibers, continuous fibers, and the like. Particularly, when it is tabular one, the thermal conductance of the cured product is increased by the lamination effect of the inorganic filler itself and the heat-releasing ability of the cured product is further improved, so that the shape is preferred.

The amount of the inorganic filler in the epoxy resin composition of the present invention is usually 2 to 1000 parts by mass. However, in order to increase the thermal conductivity as far as possible, it is preferable to increase the amount of the inorganic filler as far as possible in the range where handling and the like are not disturbed in specific uses of the epoxy resin composition of the present invention. These inorganic fillers may be used singly or two or more kinds thereof may be used in combination.

Moreover, within the range where the thermal conductivity can be maintained at 20 W/m·K or more as the whole filler, the inorganic filler having a thermal conductivity of 20 W/m·K or more may be used in combination with the inorganic filler having a thermal conductivity of less than 20 W/m·K. However, from the object of the present invention that a cured product having a high thermal conductivity as far as possible is obtained, the use of the filler having a thermal conductivity of less than 20 W/m·K should be limited to the minimum. The kind and shape of the filler usable in combination is not particularly limited.

In the case where the epoxy resin composition of the present invention is used in the semiconductor encapsulation uses, it is preferred to use the inorganic filler having a thermal conductivity of 20 W/m·K or more in a ratio of 75 to 93% by mass in the epoxy resin composition from the viewpoints of thermal resistance, moisture resistance, dynamic properties, and the like of the cured product. In this case, the remainder is the epoxy resin component, the curing agent component, and other additives to be added according to need. The additives include the other inorganic fillers which can be used in combination, a curing accelerator to be mentioned below, and the like.

A curing accelerator may be included in the epoxy resin composition of the present invention. Examples of the usable curing accelerator include imidazoles such as 2-methylimidazole, 2-ethylimidazole, 2-phenylimidazole, and 2-ethyl-4-methylimidazole; tertiary amines such as 2-(dimethylaminomethyl)phenol, triethylenediamine, triethanolamine, and 1,8-diazabicyclo(5.4.0)undecene-7; organic phosphines such as tripheylphosphine, diphenylphosphine, and tributylphosphine; metal compounds such as tin octylate; tetrasubstituted phosphonium tetrasubstituted borate such as tetraphenylphosphonium tetraphenylborate and tetraphenylphosphonium ethyltriphenylborate; tetraphenylboron salts such as 2-ethyl-4-methylimidazole tetraphenylborate and N-methylmorpholine tetraphenylborate; and the like. The curing accelerator is used in an amount of 0.01 to 15 parts by mass based on 100 parts by mass of the epoxy resin according to need.

To the epoxy resin composition of the present invention, if necessary, various blend agents such as a silane coupling agent, a releasing agent, and a pigment, various thermosetting resins, various thermoplastic resins, and the like can be added. Specific examples of the thermosetting resins and the thermoplastic resins include vinyl ester resins, unsaturated polyester resins, maleimide resins, cyanate resins, isocyanate compounds, benzoxazine compounds, vinyl benzyl ether compounds, polybutadiene and modified compounds thereof, modified compounds of acrylonitrile copolymers, indene resins, fluorocarbon resins, silicone resins, polyether imide, polyether sulfone, polyphenylene ether, polyacetal, polystyrene, polyethylene, dicyclopentadiene resins, and the like. The thermosetting resin or thermoplastic resin is used in an amount of 60% by mass or less in the epoxy resin composition of the present invention.

The epoxy resin composition of the present invention is obtained by mixing the above individual components homogeneously and preferable uses thereof include semiconductor encapsulating materials, printed wiring boards, and the like.

The epoxy resin composition of the present invention can be easily transformed into a cured product thereof by the same methods as those hitherto known. For example, the epoxy resin composition of the present invention is obtained by sufficiently mixing the epoxy resin which is an essential component of the epoxy resin composition of the present invention, the curing agent, and the inorganic filler having a thermal conductivity of 20 W/m·K or more and, if necessary, the curing accelerator, the blend agent, and various thermosetting resins and various thermoplastic resins using an extruder, a kneader, a roll, or the like according to need until a homogeneous mixture is formed and the resulting epoxy resin composition of the present invention is molded by a melt-casting method or a transfer molding method, an injection molding method, compression molding method, or the like and further heating it at a temperature of its melting point or higher for 2 to 10 hours, whereby a cured product of the epoxy resin composition of the present invention can be obtained. By encapsulating semiconductor elements mounted on a lead frame or the like by the aforementioned method, the epoxy resin composition of the present invention can be used in the semiconductor encapsulation uses.

Moreover, the epoxy resin composition of the present invention can be formed into a varnish containing a solvent. The varnish can be obtained by mixing a mixture containing at least one of the epoxy resin of the present invention and the phenolic resin of the present invention as at least one of an epoxy resin and a curing agent thereof and, if necessary, containing the other component(s) such as the inorganic filler having a thermal conductivity of 20 W/m·K or more with an organic solvent such as toluene, xylene, acetone, methyl ethyl ketone, methyl isobutyl ketone, cyclohexanone, cyclopentanone, N,N′-dimethylformamide, N,N′-dimethylacetamide, dimethyl sulfoxide, N-methylpryrrolidone, a glycol ether including ethylene glycol dimethyl ether; ethylene glycol diethyl ether; dipropylene glycol dimethyl ether; dipropylene glycol diethyl ether; triethylene glycol dimethyl ether; triethylene glycol diethyl ether; or the like, an ester including ethyl acetate; butyl acetate; methylcellosolve acetate; ethylcellosolve acetate; butylcellosolve acetate; carbitol acetate; propylene glycol monomethyl ether acetate; dialkyl glutarate; dialkyl succinate; dialkyl adipate; or the like, a cyclic ester including γ-butyrolactone or the like, or a petroleum-based solvent including petroleum ether; petroleum naphtha; hydrogenated petroleum naphtha; solvent naphtha; or the like. The amount of the solvent is usually from 10 to 95% by mass and preferably from 15 to 85% by mass based on the whole varnish.

After impregnation of a fibrous substrate such as a glass fiber, a carbon fiber, a polyester fiber, a polyamide fiber, an alumina fiber, or paper with the varnish obtained as above, the solvent is removed by heating and also the epoxy resin composition of the present invention is transformed into a semi-cured state, whereby the prepreg of the present invention can be obtained. In this regard, the “semi-cured state” herein means a state that a part of the epoxy groups that are reactive functional groups remain unreacted. A cured product can be obtained by subjecting the prepreg to hot-pressing molding.

### EXAMPLES

Although the following will explain the present invention in further detail with reference to Examples, the present invention is not limited to these Examples. Part(s) means part(s) by mass in Synthetic Examples, Examples, and Comparative Examples.

In this connection, the epoxy equivalent, melting point, softening point, total chlorine content, and thermal conductivity are measured under the following conditions.

### Epoxy Equivalent

It is measured by the method described in JIS K-7236 and the unit is g/eq.

### Melting Point

EXSTAR 6000 manufactured by Seiko Instruments Inc.

Samples to be measured: 2 mg to 5 mg, temperature-elevating rate: 10° C./min.

### Softening Point

It is measured by the method in accordance with JIS K-7234 and the unit is ° C.

### Total Chlorine Content

A value obtained by measuring an amount of chlorine (mol) released by adding a 1N—KOH propylene glycol solution to a butycarbitol solution of a sample and reflux for 10 minutes to subsequently divide the amount of chlorine by weight of the sample.

### Thermal Conductivity

It is measured by the method in accordance with ASTM E1530 and the unit is W/m·K.

### Example 1

Into a flask equipped with a stirrer, a reflux condenser, and a stirring apparatus, 136 parts of 4′-hydroxyacetophenone, 152 parts of vanillin, and 200 parts of ethanol were charged, followed by dissolving them. After adding 20 parts of 97% by mass sulfuric acid thereto, temperature was elevated to 60° C. and reaction was carried out at the temperature for 10 hours. Then, the reaction solution was poured into 1200 parts of water to precipitate crystals. After separating the crystals by filtration, they were washed with 600 parts of water twice and then dried under vacuum to obtain 256 parts of a phenolic compound 1 as yellow crystals. Endothermic peak temperature of the resulting crystals on DSC measurement was 233° C.

### Example 2

Into a flask equipped with a stirrer, a reflux condenser and a stirring apparatus, 166 parts of 4′-hydroxy-3′-methoxyacetophenone, 122 parts of 4-hydroxybenzaldehyde, and 200 parts of ethanol were charged, followed by dissolving them. After adding 20 parts of 97% by mass sulfuric acid thereto, temperature was elevated to 50° C. and reaction was carried out at the temperature for 10 hours. Then, the reaction solution was poured into 1200 parts of water to precipitate crystals. After separation of the crystals by filtration, they were washed with 600 parts of water twice and then dried under vacuum to obtain 285 parts of a phenolic compound 2 as dark brown crystals. Endothermic peak temperature of the resulting crystals on DSC measurement was 193° C.

### Example 3

Into a flask equipped with a stirrer, a reflux condenser, and a stirring apparatus, 56 parts of 4-methylcyclohexanone, 152 parts of vanillin, and 150 parts of ethanol were charged, followed by dissolving them. After adding 10 parts of 97% by mass sulfuric acid thereto, temperature was elevated to 50° C. and reaction was carried out at the temperature for 10 hours. Then, 25 parts of sodium tripolyphosphate was added and the whole was stirred for 30 minutes. Thereafter, 500 parts of methyl isobutyl ketone was added and the resulting mixture was washed with 200 parts of water twice. Then, the solvent was removed by distillation on an evaporator to obtain 304 parts of a phenolic compound 3 as a semi-solid.

### Example 4

To a flask equipped with a stirrer, a reflux condenser, and a stirring apparatus, 135 parts of the phenolic compound obtained in Example 1, 925 parts of epichlorohydrin, and 139 parts of dimethyl sulfoxide (hereinafter DMSO) were added with performing nitrogen purge, and temperature was elevated to 45° C. under stirring, followed by dissolving them. After adding 40 parts of flaky sodium hydroxide by portions over a period of 90 minutes, reaction was carried out at 45° C. for 1.5 hours and then, continued for 30 minutes after elevation of the temperature to 70° C. After completion of the reaction, 800 parts of the solvents such as excessive epichlorohydrin were removed by distillation at 70° C. under reduced pressure using a rotary evaporator. The residue was poured into 1500 parts of water to precipitate crystals. After filtration of the crystals, they were washed with 600 parts of methanol and then dried at 70° C. under vacuum to obtain 181 parts of an epoxy resin 1. Epoxy equivalent of the obtained epoxy resin was 210 g/eq. and endothermic peak temperature on DSC measurement was 118° C. and 130° C. Moreover, when total chlorine content of the obtained epoxy resin was measured, it was 1400 ppm.

### Example 5

To a flask equipped with a stirrer, a reflux condenser, and a stirring apparatus, 135 parts of the phenolic compound obtained in Example 2, 925 parts of epichlorohydrin, and 139 parts of DMSO were added with performing nitrogen purge, and temperature was elevated to 45° C. under stirring, followed by dissolving them. After adding 40 parts of flaky sodium hydroxide by portions over a period of 90 minutes, reaction was carried out at 45° C. for 1.5 hours and then, after elevation of the temperature to 70° C., the reaction was continued for 30 minutes. After completion of the reaction, 800 parts of the solvents such as excessive epichlorohydrin were removed by distillation at 70° C. under reduced pressure using a rotary evaporator. The residue was poured into 1500 parts of water to precipitate crystals. After filtration of the crystals, they were washed with 600 parts of methanol and then dried at 70° C. under vacuum to obtain 180 parts of an epoxy resin 2. Epoxy equivalent of the obtained epoxy resin was 212 g/eq. and melting point was 133° C. on DSC. Moreover, when total chlorine content of the obtained epoxy resin was measured, it was 1500 ppm.

### Example 6

To a flask equipped with a stirrer, a reflux condenser, and a stirring apparatus, 160 parts of the phenolic compound obtained in Example 3, 925 parts of epichlorohydrin, and 139 parts of DMSO with performing nitrogen purge were added and temperature was elevated to 45° C. under stirring, followed by dissolving them. After adding 40 parts of flaky sodium hydroxide by portions over a period of 90 minutes, reaction was carried out at 45° C. for 1.5 hours and then, after elevation of the temperature to 70° C., the reaction was continued for 30 minutes. After completion of the reaction, 800 parts of the solvents such as excessive epichlorohydrin were removed by distillation at 70° C. under reduced pressure using a rotary evaporator. The residue was poured into 1500 parts of water to precipitate crystals. After filtration of the crystals, they were washed with 600 parts of methanol and then were dried at 70° C. under vacuum to obtain 199 parts of an epoxy resin 3. Epoxy equivalent of the obtained epoxy resin was 298 g/eq. and melting point was 119° C. on DSC. Moreover, when total chlorine content of the obtained epoxy resin was measured, it was 1450 ppm.

### Examples 7 to 15 and Comparative Examples 1 and 2

Various components were blended in ratios (parts) described in Table 1 and each of the resulting blends was kneaded in a mixing roll and formed into tablets. Thereafter, the tablets were subjected to transfer molding to prepare a resin molded article, which was then heated at 160° C. for 2 hours and further at 180° C. for 8 hours to obtain each of cured products of epoxy resin compositions of the present invention and resin compositions for comparison. The results of measurement of thermal conductivity of the cured products are shown in Table 1.

TABLE 1

| | Exam- | Exam- | Exam- | Exam- | Exam- | Exam- | Exam- | Exam- | Exam- | Comparative | Comparative |
|---|---|---|---|---|---|---|---|---|---|---|---|
| | ple 7 | ple 8 | ple 9 | ple 10 | ple 11 | ple 12 | ple 13 | ple 14 | ple 15 | Example 1 | Example 2 |
| Composition of | | | | | | | | | | | |
| blend | | | | | | | | | | | |
| Epoxy resin 1 | 100 | | | | | | 100 | | | | |
| Epoxy resin 2 | | 100 | | | | | | 100 | | | |
| Epoxy resin 3 | | | 100 | | | | | | 100 | | |
| Epoxy resin 4 | | | | 100 | 100 | 100 | | | | 100 | |
| Epoxy resin 5 | | | | | | | | | | | 100 |
| Curing agent 1 | | | | 49 | | | 64 | | | | |
| Curing agent 2 | | | | | 49 | | | 64 | | | |
| Curing agent 3 | | | | | | 69 | | | 64 | | |
| Curing agent 4 | 50 | 50 | 35 | | | | | | | 38 | 60 |
| Curing | 1 | 1 | 1 | 1 | 1 | 1 | 1 | 1 | 1 | 1 | 1 |
| accelerator | | | | | | | | | | | |
| Physical property | | | | | | | | | | | |
| of cured product | | | | | | | | | | | |
| Thermal | 0.27 | 0.28 | 0.29 | 0.26 | 0.27 | 0.28 | 0.30 | 0.31 | 0.35 | 0.25 | 0.26 |
| conductivity | | | | | | | | | | | |
| (W/m · K) | | | | | | | | | | | |

Epoxy resin 4: Epoxy resin represented by the following formula (7) (trade name: NC-3000 manufactured by Nippon Kayaku Co., Ltd., epoxy equivalent: 276 g/eq.)

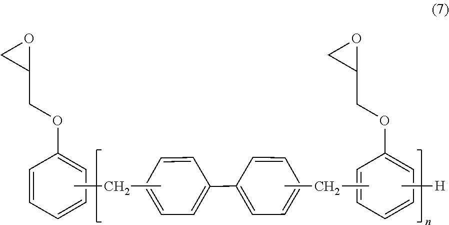

Epoxy resin 5: Biphenyl-type epoxy resin containing epoxy resins represented by the following formulae (8) and (9) in equimolar amounts (trade name: YL-6121H manufactured by Japan Epoxy Resin, epoxy equivalent: 175 g/eq.)

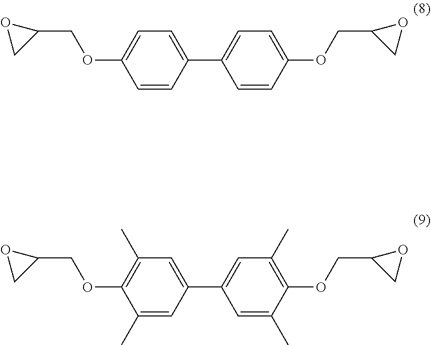

Curing agent 1: Phenolic compound 1 obtained in Example 1

Curing agent 2: Phenolic compound 2 obtained in Example 2

Curing agent 3: Phenolic compound 3 obtained in Example 3

Curing agent 4: Phenol novolak represented by the following formula (10) (trade name: H-1, manufactured by Meiwa Kasei, hydroxyl equivalent: 105 g/eq.)

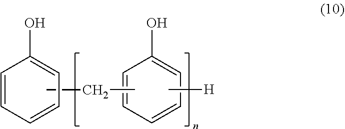

Curing accelerator: Triphenylphosphine (manufactured by Hokko Chemical Industry Co., Ltd.)

### Examples 16 to 24 and Comparative Examples 3 and 4

Various components were blended in ratios (parts) of Table 2, followed by kneading in a mixing roll to be formed into tablets. Thereafter, the tablets were subjected to transfer molding to prepare a resin molded article, which was then heated at 160° C. for 2 hours and further at 180° C. for 8 hours to obtain each of cured products of epoxy resin compositions of the present invention and resin compositions for comparison. The results of measurement of thermal conductivity of these cured products are shown in Table 2.

TABLE 2

| | Exam- | Exam- | Exam- | Exam- | Exam- | Exam- | Exam- | Exam- | Exam- | Comparative | Comparative |
|---|---|---|---|---|---|---|---|---|---|---|---|
| | ple 16 | ple 17 | ple 18 | ple 19 | ple 20 | ple 21 | ple 22 | ple 23 | ple 24 | Example 3 | Example 4 |
| Composition of | | | | | | | | | | | |
| blend | | | | | | | | | | | |
| Epoxy resin 1 | 100 | | | | | | 100 | | | | |
| Epoxy resin 2 | | 100 | | | | | | 100 | | | |
| Epoxy resin 3 | | | 100 | | | | | | 100 | | |
| Epoxy resin 4 | | | | 100 | 100 | 100 | | | | 100 | |
| Epoxy resin 5 | | | | | | | | | | | 100 |
| Curing agent 1 | | | | 49 | | | 64 | | | | |
| Curing agent 2 | | | | | 49 | | | 64 | | | |
| Curing agent 3 | | | | | | 69 | | | 64 | | |
| Curing agent 4 | 50 | 50 | 35 | | | | | | | 38 | 60 |
| Curing | 1 | 1 | 1 | 1 | 1 | 1 | 1 | 1 | 1 | 1 | 1 |
| accelerator | | | | | | | | | | | |
| Inorganic filler 1 | 342 | 342 | 308 | 340 | 340 | 386 | 375 | 375 | 375 | 316 | 366 |
| Inorganic filler 2 | 195 | 195 | 178 | 194 | 194 | 220 | 213 | 213 | 213 | 180 | 208 |
| Physical property | | | | | | | | | | | |
| of cured product | | | | | | | | | | | |
| Thermal | 3.8 | 3.9 | 4.0 | 3.7 | 3.8 | 3.9 | 4.1 | 4.3 | 4.8 | 3.3 | 3.5 |
| conductivity | | | | | | | | | | | |
| (W/m · K) | | | | | | | | | | | |

Inorganic filler 1: Spherical alumina (trade name: DAW-100 manufactured by Denki Kagaku Kogyo Kabushiki Kaisha, thermal conductivity: 38 W/m·K)

Inorganic filler 2: Boron nitride (trade name: SGP manufactured by Denki Kagaku Kogyo Kabushiki Kaisha, thermal conductivity: 60 W/m·K)

### Example 25

After dissolving 100 parts of the epoxy resin 3 obtained in Example 6 in 1000 parts of dimethylformamide at 70° C., the temperature was returned to room temperature. After dissolving 13 parts of 1,5-naphthalenediamine (manufactured by Tokyo Chemical Industry Co., Ltd., amine equivalent: 40 g/eq.) as a curing agent in 48 parts of dimethylformamide at 70° C., the temperature was returned to room temperature. The above epoxy resin solution and curing agent solution were mixed and stirred in a stirring blade-type homomixer to form a homogeneous varnish. Furthermore, 215 parts of the inorganic filler (trade name: SGP manufactured by Denki Kagaku Kogyo Kabushiki Kaisha, thermal conductivity: 60 W/m·K) (50 parts by volume based on 100 parts by volume of resin solid matter) and 100 parts of dimethylformamide were added thereto and the whole was mixed and stirred to prepare an epoxy resin composition of the present invention.

A glass fiber woven fabric (trade name: 7628/AS890AW manufactured by Asahi-Schwebel Co., Ltd.) having a thickness of 0.2 mm was impregnated with the varnish of the epoxy resin composition and then heated and dried to obtain a prepreg. After overlaying four sheets of the prepreg and copper foils placed to both sides thereof, they were integrated by molding though heating and pressurization under conditions of a temperature of 175° C. and a pressure of 4 MPa for 90 minutes to obtain a laminated board having a thickness of 0.8 mm. When thermal conductivity of the laminated board was measured, it was 4.9 W/m·K.

### Example 26

After dissolving 100 parts of the epoxy resin 4 (NC-3000) and 69 parts of the phenolic compound 3 obtained in Example 3 in 1000 parts of dimethylformamide at 70° C., the temperature was returned to room temperature. After dissolving 1 part of triphenylphosphine (manufactured by Hokko Chemical Industry Co., Ltd.) as a curing accelerator in 48 parts of dimethylformamide at 70° C., the temperature was returned to room temperature. The above epoxy resin solution and curing accelerator solution were mixed and stirred in a stirring blade-type homomixer to form a homogeneous varnish. Furthermore, 321 parts of the inorganic filler (trade name: SGP manufactured by Denki Kagaku Kogyo Kabushiki Kaisha, thermal conductivity: 60 W/m·K) (50 parts by volume based on 100 parts by volume of resin solid matter) and 100 parts of dimethylformamide were added thereto and the whole was mixed and stirred to prepare an epoxy resin composition of the present invention.

A glass fiber woven fabric (trade name: 7628/AS890AW manufactured by Asahi-Schwebel Co., Ltd.) having a thickness of 0.2 mm was impregnated with the varnish of the epoxy resin composition and then heated and dried to obtain a prepreg. After overlaying four sheets of the prepreg and copper foils placed to both sides thereof, they were integrated by molding though heating and pressurization under conditions of a temperature of 175° C. and a pressure of 4 MPa for 90 minutes to obtain a laminated board having a thickness of 0.8 mm. When thermal conductivity of the laminated board was measured, it was 4.7 W/m·K.

### Comparative Example 5

A laminated board was obtained in the same operation procedure as in Example 25 except that the epoxy resin 3 in Example 25 was changed to 100 parts of the epoxy resin 5 (YL-6121H); the amount of 1,5-naphthalenediamine was changed to 23 parts; and the amount of the inorganic filler was changed to 234 parts. When thermal conductivity of the laminated board was measured, it was 3.6 W/m·K.

### Comparative Example 6

A laminated board was obtained in the same operation procedure as in Example 26 except that 69 parts of the phenolic compound 3 in Example 26 was changed to 29 parts of the phenol novolak resin represented by the formula (10) and the amount of the inorganic filler was changed to 245 parts. When thermal conductivity of the laminated board was measured, it was 3.9 W/m·K.

### Example 27

Into a flask equipped with a stirrer, a reflux condenser, and a stirring apparatus, 29 parts of acetone, 152 parts of vanillin, and 300 parts of ethanol were charged, followed by dissolving them. After adding 80 parts of a 50% aqueous sodium hydroxide solution, temperature was elevated to 45° C. and reaction was carried out at the temperature for 120 hours. Then, the reaction solution was poured into 800 mL of 1.5N hydrochloric acid to precipitate crystals. After separation of the crystals by filtration, they were washed with 600 parts of water twice and then dried under vacuum to obtain 165 parts of a phenolic compound 4 as yellow crystals. Melting point of the resulting crystals was 201° C. on DSC measurement.

### Example 28

To a flask equipped with a stirrer, a reflux condenser, and a stirring apparatus, 163 parts of the phenolic compound 4 obtained in Example 27, 925 parts of epichlorohydrin, and 139 parts of DMSO were added with performing nitrogen purge, and temperature was elevated to 45° C. under stirring, followed by dissolving them. After adding 40 parts of flaky sodium hydroxide by portions over a period of 90 minutes, reaction was carried out at 45° C. for 1.5 hours and then, after elevation of the temperature to 70° C., continued for 30 minutes. After completion of the reaction, 800 parts of the solvents such as excessive epichlorohydrin were removed by distillation at 70° C. under reduced pressure using a rotary evaporator. The residue was poured into 1500 parts of water to precipitate crystals. After filtration of the crystals, they were washed with 600 parts of methanol and then dried at 70° C. under vacuum to obtain 200 parts of an epoxy resin 6. Epoxy equivalent of the obtained epoxy resin was 256 g/eq., melting point was 140° C. on DSC measurement. Moreover, when total chlorine content of the obtained epoxy resin was measured, it was 1400 ppm.

### Example 29

To a flask equipped with a stirrer, a reflux condenser, and a stirring apparatus, 135 parts of the phenolic compound 1 obtained in Synthetic Example 1, 278 parts of epichlorohydrin, 93 parts of dimethyl sulfoxide, and 6 parts of water were added with performing nitrogen purge, and temperature was elevated to 40° C. under stirring. After adding 42 parts of flaky sodium hydroxide by portions over a period of 90 minutes, reaction was carried out with stirring at 40° C. for 2 hours; at 50° C. for 2 hours; and at 70° C. for 1 hour. After completion of the reaction, excessive epichlorohydrin, dimethyl sulfoxide, and the like were removed by distillation from an oily layer at 130° C. under reduced pressure using a rotary evaporator. Then, 473 parts of methyl isobutyl ketone was added to the residue and it was dissolved, followed by elevating the temperature to 70° C. After washing the solution with water to remove salts, the temperature was again elevated to 70° C. and 11 parts of a 30% by weight aqueous sodium hydroxide solution was added thereto under stirring, followed by carrying out reaction for 1 hour. Thereafter, washing with water was performed until the washing water became neutral and methyl isobutyl ketone and the like were removed by distillation from the resulting solution at 180° C. under reduced pressure using a rotary evaporator to obtain 173 parts of an objective epoxy resin 7. Epoxy equivalent of the obtained epoxy resin was 236 g/eq.; JIS softening point was 63° C.; and total chlorine content was 550 ppm.

### Example 30

To a flask equipped with a stirrer, a reflux condenser, and a stirring apparatus, 135 parts of the phenolic compound 1 obtained in Synthetic Example 1, 278 parts of epichlorohydrin, and 28 parts of methanol were added with performing nitrogen purge, and temperature was elevated to 70° C. under stirring to dissolve them. After adding 42 parts of flaky sodium hydroxide by portions over a period of 90 minutes, reaction was carried out at 70° C. for 1.5 hours. After completion of the reaction, washing with water was performed and then excessive epichlorohydrin and the like were removed by distillation from an oily layer at 130° C. under reduced pressure using a rotary evaporator. Then, 382 parts of methyl isobutyl ketone was added to the residue to dissolve it, followed by elevating the temperature to be 70° C. Then, 12 parts of a 30% by weight aqueous sodium hydroxide solution was added thereto under stirring and reaction was carried out at 70° C. for 75 minutes. Thereafter, washing with water was performed until the washing water became neutral and methyl isobutyl ketone and the like were removed by distillation from the resulting solution at 180° C. under reduced pressure using a rotary evaporator to obtain 175 parts of an objective epoxy resin 8. Epoxy equivalent of the obtained epoxy resin was 225 g/eq.; JIS softening point was 55° C.; and total chlorine content was 600 ppm.

### REFERENCE EXAMPLE

in which aqueous sodium hydroxide solution was used

To a flask equipped with a stirrer, a reflux condenser, and a stirring apparatus 135 parts of the phenolic compound 1 obtained in Example 1 and 231 parts of epichlorohydrin were added with performing nitrogen purge. When temperature was elevated to 90° C. under stirring, 125 parts of a 16% aqueous sodium hydroxide solution was added thereto and then the whole was stirred at 90° C. for 40 minutes. Thereafter, 25 parts of a 40% aqueous sodium hydroxide solution was further added thereto and reaction was carried out for 20 minutes. After completion of the reaction, washing with water was performed and then excessive epichlorohydrin and the like were removed by distillation at 135° C. under reduced pressure using a rotary evaporator. After dissolving the residue in 382 parts of methyl isobutyl ketone, washing with water was again performed and then low-boiling components such as methyl isobutyl ketone were removed by distillation at 180° C. under reduced pressure using a rotary evaporator to obtain 181 parts of an epoxy resin 9. Epoxy equivalent of the obtained epoxy resin was 270 g/eq. and softening point was 68° C. Moreover, when total chlorine content of the obtained epoxy resin was measured, it was 5000 ppm or more.

Solubility of various epoxy resins including the epoxy resins 1 to 3 and 6 to 8 obtained by these operations in methyl isobutyl ketone at 60° C. and 100° C. was shown in Table 3.

TABLE 3

| | |
|---|---|
| Epoxy resin 1 | ◯ |
| Epoxy resin 2 | ◯ |
| Epoxy resin 3 | ◯ |
| Epoxy resin 4 | ⊚ |
| Epoxy resin 5 | X |
| Epoxy resin 6 | ◯ |
| Epoxy resin 7 | ⊚ |
| Epoxy resin 8 | ⊚ |
| Epoxy resin 9 | ⊚ |

⊚ . . . dissolved at 60° C.
◯ . . . dissolved at 100° C.
X . . . not completely dissolved at 100° C. (insoluble residue is present)

### Examples 31 to 37, Comparative Examples 1 and 27, and Reference Example 1

Various components were blended in ratios (parts) of Table 4 followed by kneading in a mixing roll to be formed into tablets. Thereafter, the tablets were subjected to transfer molding to prepare a resin molded article, which was then heated at 160° C. for 2 hours and further at 180° C. for 8 hours to obtain each of cured products of epoxy resin compositions of the present invention and resin compositions for comparison. The results of measurement of thermal conductivity of these cured products are shown in Table 4.

TABLE 4

| | Exam- | Exam- | Exam- | Exam- | Exam- | Exam- | Exam- | Comparative | Comparative | Reference |
|---|---|---|---|---|---|---|---|---|---|---|
| | ple 31 | ple 32 | ple 33 | ple 34 | ple 35 | ple 36 | ple 37 | Example 1 | Example 2 | Example 1 |
| Composition of | | | | | | | | | | |
| blend | | | | | | | | | | |
| Epoxy resin 6 | 100 | | | | 100 | | | | | |
| Epoxy resin 7 | | 100 | | | | 100 | | | | |
| Epoxy resin 8 | | | 100 | | | | 100 | | | |
| Epoxy resin 4 | | | | 100 | | | | 100 | | |
| Epoxy resin 5 | | | | | | | | | 100 | |
| Epoxy resin 9 | | | | | | | | | | 100 |
| Curing agent 1 | | | | | | 57 | 60 | | | |
| Curing agent 4 | 41 | 44 | 47 | | | | | 38 | 60 | 39 |
| Curing agent 5 | | | | 59 | 64 | | | | | |
| Curing | 1 | 1 | 1 | 1 | 1 | 1 | 1 | 1 | 1 | 1 |
| accelerator | | | | | | | | | | |
| Physical property | | | | | | | | | | |
| of cured product | | | | | | | | | | |
| Thermal | 0.27 | 0.29 | 0.29 | 0.27 | 0.30 | 0.32 | 0.32 | 0.25 | 0.26 | 0.24 |
| conductivity | | | | | | | | | | |
| (W/m · K) | | | | | | | | | | |

Curing agent 5: Phenolic compound 4 obtained in Example 27

### Examples 38 to 44, Comparative Examples 3 and 4, and Reference Example 2

Various components were blended in ratios (parts) of Table 5, followed by kneading in a mixing roll to be formed into tablets. Thereafter, the tablets were subjected to transfer molding to prepare a resin molded article, which was then heated at 160° C. for 2 hours and further at 180° C. for 8 hours to obtain each of cured products of epoxy resin compositions of the present invention and resin compositions for comparison. The results of measurement of thermal conductivity of these cured products are shown in Table 5.

TABLE 5

| | Exam- | Exam- | Exam- | Exam- | Exam- | Exam- | Exam- | Comparative | Comparative | Reference |
|---|---|---|---|---|---|---|---|---|---|---|
| | ple 38 | ple 39 | ple 40 | ple 41 | ple 42 | ple 43 | ple 44 | Example 3 | Example 4 | Example 2 |
| Composition of | | | | | | | | | | |
| blend | | | | | | | | | | |
| Epoxy resin 6 | 100 | | | | 100 | | | | | |
| Epoxy resin 7 | | 100 | | | | 100 | | | | |
| Epoxy resin 8 | | | 100 | | | | 100 | | | |
| Epoxy resin 4 | | | | 100 | | | | 100 | | |
| Epoxy resin 5 | | | | | | | | | 100 | |
| Epoxy resin 9 | | | | | | | | | | 100 |
| Curing agent 1 | | | | | | 57 | 60 | | | |
| Curing agent 4 | 41 | 44 | 47 | | | | | 38 | 60 | 39 |
| Curing agent 5 | | | | 59 | 64 | | | | | |
| Curing | 1 | 1 | 1 | 1 | 1 | 1 | 1 | 1 | 1 | 1 |
| accelerator | | | | | | | | | | |
| Inorganic filler 1 | 323 | 330 | 337 | 364 | 375 | 360 | 366 | 316 | 366 | 318 |
| Inorganic filler 2 | 183 | 187 | 192 | 207 | 213 | 204 | 208 | 180 | 208 | 181 |
| Physical property | | | | | | | | | | |
| of cured product | | | | | | | | | | |
| Thermal | 3.7 | 3.9 | 3.9 | 3.7 | 4.1 | 4.3 | 4.3 | 3.3 | 3.5 | 3.4 |
| conductivity | | | | | | | | | | |
| (W/m · K) | | | | | | | | | | |

From the above results, it could be confirmed that the epoxy resin of the present invention is excellent in solvent solubility and also the cured product of the epoxy resin composition containing at least one of the phenolic compound and the epoxy resin of the present invention has an excellent thermal conductance. Particularly, it could be confirmed that the total chlorine content is reduced and a good thermal conductivity is exhibited by the use of flaky sodium hydroxide as the alkali metal hydroxide at epoxidation. Moreover, by performing addition thereof by portions, the formation of 1,3-halohydrin compound and halomethylene compound which are impurities can be prevented and thermal conductivity can be improved.

Therefore, the phenolic compound and the epoxy resin of the present invention are extremely useful in the case where they are used in insulating materials, laminated boards (printed wiring boards and the like), and the like for electric/electronic parts.

While the present invention has been described in detail and with reference to specific embodiments thereof, it will be apparent to one skilled in the art that various changes and modifications can be made therein without departing from the spirit and scope thereof.

The present application is based on Japanese Patent Application No. 2010-019269 filed on Jan. 29, 2010, and the contents are incorporated herein by reference. Also, all the references cited herein are incorporated as a whole.

### INDUSTRIAL APPLICABILITY

The cured product of the epoxy resin composition of the present invention has an excellent thermal conductance in comparison with cured products of conventional epoxy resins and also is excellent in solvent solubility. Therefore, the cured product is extremely useful as an encapsulating material, a prepreg, and the like in a wide range of uses such as electric/electronic materials, molding materials, casting materials, lamination materials, paints, adhesives, resists, and optical materials.

1. (1) A phenolic compound obtained by reacting one or more compounds represented by the following formulae (1) to (5): 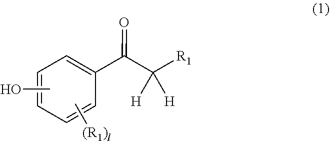
wherein R₁ groups are each independently present and represent any of a hydrogen atom, a substituted or unsubstituted alkyl group having 1 to 10 carbon atoms, a substituted or unsubstituted aryl group having 6 to 10 carbon atoms, a hydroxyl group, a nitro group, or a substituted or unsubstituted alkoxy group having 1 to 10 carbon atoms and 1 represents the number of R₁ groups and is an integer of 0 to 4; 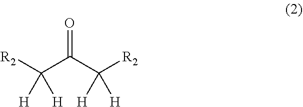
wherein R₂ groups are each independently present and represent any of a hydrogen atom, a substituted or unsubstituted alkyl group having 1 to 20 carbon atoms, a substituted or unsubstituted aryl group having 6 to 10 carbon atoms, a substituted or unsubstituted alkylcarbonyl group having 1 to 15 carbon atoms, a substituted or unsubstituted alkyl ester group having 2 to 10 carbon atoms, a substituted or unsubstituted alkoxy group having 1 to 10 carbon atoms, a morpholinylcarbonyl group, a phthalimido group, a piperonyl group, or a hydroxyl group; 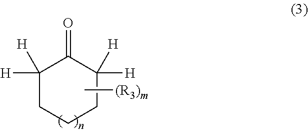
wherein R₃ groups are each independently present and represent any of a hydrogen atom, a substituted or unsubstituted alkylcarbonyl group having 0 to 10 carbon atoms, a substituted or unsubstituted alkyl group having 1 to 10 carbon atoms, a substituted or unsubstituted aryl group having 6 to 10 carbon atoms, a substituted or unsubstituted alkyl ester group having 2 to 10 carbon atoms, a substituted or unsubstituted alkoxy group having 1 to 10 carbon atoms, or a hydroxyl group; n represents the number of carbon atoms and is any integer of 0, 1 and; and m represents the number of R₃ groups and satisfies the relation: 0≦m≦n+2; 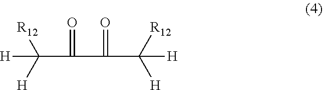
wherein R₁₂ groups are each independently present and represent any of a hydrogen atom, a substituted or unsubstituted alkyl group having 1 to 20 carbon atoms, a substituted or unsubstituted aryl group having 6 to 10 carbon atoms, a substituted or unsubstituted alkoxy group having 1 to 10 carbon atoms, or a hydroxyl group; 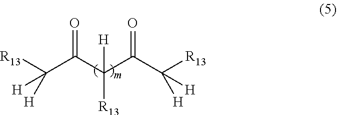
wherein R₁₃ groups are each independently present and represent any of a hydrogen atom, a substituted or unsubstituted alkyl group having 1 to 20 carbon atoms, a substituted or unsubstituted aryl group having 6 to 10 carbon atoms, a substituted or unsubstituted alkoxy group having 1 to 10 carbon atoms, a substituted or unsubstituted alkyl ester group having 1 to 10 carbon atoms, or a hydroxyl group; and m is an integer of 1 to 10;
with the following formula (6): 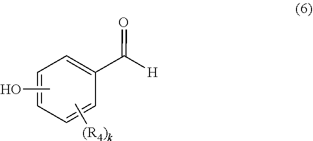
wherein R₄ groups are each independently present and represent any of a hydrogen atom, a substituted or unsubstituted alkyl group having 1 to 10 carbon atoms, a substituted or unsubstituted aryl group having 6 to 10 carbon atoms, a hydroxyl group, a nitro group, a formyl group, an allyl group, or a substituted or unsubstituted alkoxy group having 1 to 10 carbon atoms; and k represents the number of R₄ groups and is an integer of 0 to 4.

2. An epoxy resin obtained by reacting the phenolic compound described in claim 1 with an epihalohydrin.

3. The epoxy resin according to claim 2, wherein total halogen content is 1800 ppm or less.

4. An epoxy resin composition comprising at least one of the epoxy resin described in claim 2 and the phenolic compound described in claim 1.

5. The epoxy resin composition according to claim 4, which contains an inorganic filler having a thermal conductivity of 20 W/m·K or more.

6. The epoxy resin composition according to claim 4, which is used in semiconductor encapsulation uses.

7. A prepreg comprising the epoxy resin composition described in claim 4 and a sheet-shaped fiber substrate.

8. A cured product obtained by curing the epoxy resin composition described in claim 4.

9. A process for producing the epoxy resin composition according to claim 3, wherein flaky sodium hydroxide is added into a reaction system at the reaction of the phenolic compound with the epihalohydrin.

10. The process according to claim 9, wherein the flaky sodium hydroxide is added into the reaction system for multiple times.

11. The process according to claim 9, wherein the epihalohydrin is used in an amount of 2 to 15 mol based on 1 mol of the hydroxyl group of the phenolic compound.

12. The process according to claim 9, wherein the epihalohydrin is used in an amount of 2 to 4.5 mol based on 1 mol of the hydroxyl group of the phenolic compound.

13. A cured product obtained by curing the prepreg according to claim 7.
